(19) United States
(12) Patent Application Publication
(10) Pub. No.: US 2003/0147835 A1
(43) Pub. Date: Aug. 7, 2003

(54) Process for the manufacture of hydrogel compositions and hydrogel compositions manufactured thereby

(76) Inventors: Hugh Semple Munro, Chipping Camden (GB); Michael Joseph Donnelly, Coventry (GB); Alison Page, Coventry (GB)

Correspondence Address: NIXON PEABODY LLP, ATTENTION: DAVID RESNICK, 101 FEDERAL STREET, BOSTON, MA, 02110, US

(21) Appl. No.: 10/311,496

(22) PCT Filed: Jun. 15, 2001
(86) PCT No.: PCT/GB01/02647

(30) Foreign Application Priority Data
Jun. 15, 2000 (GB) 0014677.9
Jul. 4, 2000 (GB) 0016450.9

(51) Int. Cl. A61K 7/06; A61K 7/11; C08F 2/46

(52) U.S. Cl. 424/70.16; 522/182

(57) ABSTRACT

The present invention provides a process for the manufacture of a high water content (greater than about 40% by weight of water) cross-linked hydrogel composition which has an acceptable non-greasy “feel”, sufficient structural integrity and low brittleness, as well as good processing properties. In the process, a photopolymerisable monomer is photopolymerised, in the presence of water, low levels of a multifunctional photopolymerisable cross-limiting agent (less than about 0.5% by weight of the total polymerisation reaction mixture) and low levels of a photoinitiator (between about 0.002% and about 0.05% by weight of the total polymerisation reaction mixture).

### FIELD OF THE INVENTION

The present invention relates to a process for the manufacture of hydrogel compositions, and more particularly to a process for the manufacture of high water content hydrogel compositions suitable for use in wound and burn dressings, biomedical electrodes and other applications where bioadhesion is required. The invention also relates to hydrogel compositions manufactured by the said process.

### BACKGROUND OF THE INVENTION

High water content cross-linked hydrogels based on hydrophilic acrylamido polymers have been previously described by Laskey (U.S. Pat. No. 3,929,741). The ability of these materials to imbibe large quantities of aqueous liquid (e.g. water, biological fluids) and to retain their structural integrity was considered to be advantageous in a-number of biomedical and consumer applications (Laskey, column 3, lines 36 to 57). Wound and burn coverings are mentioned among many other uses, but with no particular preference expressed for them. A number of methods for initiating the polymerisation and cross-linking were mentioned, including the use of irradiation (Laskey, column 5, lines 1 to 12). Details of how to achieve irradiation-initiated polymerisation and cross-linking were not described

The manufacturing processes described by Laskey suffers from a number of inherent disadvantages. Primarily, the batchwise polymerisation with cross-linking is not suitable for mass-production and the relatively high levels of cross-linking agent required (greater than about 0.5% by weight) result in a somewhat brittle hydrogel which is difficult to handle.

It is therefore an object of the present invention to provide an improved or at least alternative method for the commercial manufacture of high water content hydrogels, which yields a product having acceptable skin feel and adhesion properties for biomedical use.

### BRIEF DESCRIPTION OF THE INVENTION

The present invention is based on our surprising finding that reduced levels of cross-linking agent (less than about 0.5% by weight) can give rise to a polymerised and cross-linked hydrogel which has an acceptable non-greasy “feel”, sufficient structural integrity and low brittleness, as well as good processing properties, if the levels of photoinitiator are also reduced to substantially lower levels than used hitherto.

According to a first aspect of the present invention, there is provided a process for the manufacture of a high water content cross-led hydrogel composition, comprising:

(i) preparing a mixture comprising:

(1) one or more unsaturated free radically photo-polymerisable monomer capable of polymerisation to a hydrophilic polymer,

(2) one or more free radical photoinitiator;

(3) one or more cross-linking agent comprising a multifunctional unsaturated free radically photo-polymerisable compound; and

(4) water; and

(ii) irradiating the mixture with light of sufficient intensity and at an appropriate wavelength to polymerise and cross-link the mixture to form the composition;

wherein substantially all of components (1) to (4) present in the mixture in step (i) are also present in the composition resulting from step (i), the photoinitiator (2) is present in the mixture in step (i) in an amount between about 0.002% and about 0.05% by weight of the total mixture, and the cross-linking agent (3) is present in the mixture in step (i) in an amount less than about 0.5% by weight of the total mixture.

Most preferably, the mixture prepared in step (i) consists essentially of components (1) to (4) and optionally one or more electrolyte and/or one or more organic plasticiser and/or one or more surfactant, with less than about 10% of other additives.

It has been found that the process of the present invention is highly convenient for an industrial scale continuous sheet production method for preparing hydrogel sheets having a thickness in the range of about 0.2 mm to about 2 mm. Such sheets are typically prepared in contact with a release sheet, for example a sheet of plastic or coated plastic (e.g. siliconised plastic) or paper or coated paper (e.g. siliconised paper) at a surface weight of hydrogel in the range of about 0.5 kg/m² to about 2.5 kg/m². Moreover, the process enables high water content cross-linked hydrogel compositions having predetermined combinations of certain important characteristics-to be prepared reproducibly on an industrial scale, when the amounts of the photoinitiator and the cross-linker are controlled in the reaction mixture. According to a further aspect, the present invention maybe stated to provide the use of a controlled amount of one or more free radical photoinitiator in the range between about 0.002% and about 0.05% by weight of the total mixture and a controlled amount of one or more multifunctional unsaturated free radically photo-polymerisable cross-linking agent in the range less than about 0.5% by weight of the total mixture, in a pre-photopolymerisation mixture comprising also water and one or more unsaturated free radically photopolymerisable monomer in a process for the manufacture of a high water content cross-linked hydrogel composition having a desired pre-determined combination of two or more of: non-greasy feel to human skin, structural integrity, low brittleness and good industrial processability in sheet form.

According to a flier aspect of the present invention, there is provided a high water content cross-linked hydrogel composition manufactured by the process or use of the present invention. The composition may suitably be present in the form of a sheet having first and second major faces, each of said first and second major faces being in contact with a protective release layer, for example siliconised plastic or paper.

Alternatively, the composition may be present in the form of a sheet having first and second major faces, one of said first and second major faces being in contact with a protective release layer, for example siliconised plastic or paper, and the other of said first and second major faces being in contact with a backing member, suitably a backing member forming part of a wound or bum dressing, a biomedical electrode or another article where a bioadhesive hydrogel layer is to be provided in use between the article and the skin of a patient. Still further, the composition may be present in the form of a sheet having a woven or non-woven fabric, or a net, embedded therein.

The resultant hydrogels are low or moderately cross-linked materials which surprisingly, have a very acceptable toughness, low brittleness and high structural-integrity with generally very good machine cutting/converting performance in rolls of sheet hydrogel to be formed in excess of 100 m length. In such a process, the irradiation step (ii) will preferably take place with relative movement between an irradiation source and the mixture resulting from step (i), i.e. preferably the complete mixtures will not be irradiated simultaneously.

The expression “high water content” used herein refers particularly to hydrogel compositions comprising more than about 40% by weight of water, more particularly above about 50% by weight, and most preferably between about 60% and about 95% by weight.

The expression “monomer” used herein includes ionic and non-ionic monomers and monomermixtures. Correspondingly, the-expression “polymerise”, “polymers” and like expressions include both homopolymerisation and copolymerisation, and the products thereof.

The expression “substantially all of components (1) to (4) present in the mixture in step (i) are also present in the composition resulting from step (ii)” used herein is to be understood as meaning that the desired extent of conversion of monomer and cross-linking agent into polymer takes place, but that material is not substantially removed in or by step (ii). It is preferred also that material is not substantially added to or removed from the composition after step (ii), although some-degree of conditioning and/or modification may be desirable.

### DETAILED DESCRIPTION OF THE INVENTION

Ionic Monomer

Preferably the one or more ionic monomers are 2-acrylamido-2-methylpropane sulphonic acid or an analogue thereof or one of its salts, e.g. an ammonium or alkali metal salt such as a sodium, potassium or lithium salts; acrylic acid or an analogue thereof or one of its salts, e.g. an a metal salt such as a sodium, potassium or lithium salt; and/or a polymerisable sulphonate or a salt thereof e.g. an metal salt such as a sodium, potassium or lithium salt, of acrylic acid (3-sulphopropy! or an analogue thereof. The term “analogue” in this context refers particularly to substituted derivatives of 2-acrylamido-2-methylpropane sulphonic acid, of acrylic acid or of acrylic acid (3-sulphopropyl) ester.

A particularly preferred ionic monomer is a sodium, salt of 2-acrylamido-2-methylpropane sulphonic acid, commonly known as NaAMPS, which is available commercially at present from Lubrizol as either a 50% aqueous solution (reference code LZ2405) or a 58% aqueous solution (reference code LZ2405A) and/or acrylic acid (3-sulphopropyl) ester potassium salt, commonly known as SPA. SPA is available commercially in the form of a pure solid from Raschig. The reaction mixture preferably comprises from about 5% to about 40%, and ideally from about 10% to about 40%, by weight of the reaction mixture, of the ionic monomer, with the proviso that the total amount of monomer (1) in the reaction mixture should preferably be in the range of about 5% to about 40% by weight of the total reaction mixture.

Non-Ionic Monomer

In one embodiment of the invention the aforesaid non-ionic water soluble monomer will comprise at least one of acrylamide or a mono- or di-N-alkylacrylamide or an analogue thereof. The term “analogue” in this context refers to non-ionic water soluble monomers containing an alkyl or substituted alkyl group linked to a carbon-carbon double bond via an amido or alkylamido (—CO.NH— or —CO.NR—) function. Examples of such analogues include diacetone acrylamide (N-1,1-dimethyl-3-oxobutyl-acrylamide), vinyl lactams, N-alkylated acrylamides, N,N-dialkylated acrylamides, N-vinyl pyrrolidone and acryloyl morpholine. N,N-dimethylacrylamide (14A) and/or an analogue thereof is preferred. When present, the non-ionic water soluble monomer may comprise up to about 30%, and ideally up to about 25%, by weight of the reaction mixture, with the proviso that the total amount of monomer (1) in the reaction mixture should preferably be in the range of about 5% to about 40% by weight of the total reaction mixture.

Cross-Linking Agents and Photoinitiators

Conventional cross-agents are suitably used to provide the necessary mechanical stability and to control the adhesive properties of the hydrogel. The amount of cross-linking agent required will be readily apparent to those skilled in the art such as from about 0.01% to about 0.5%, particularly from about 0.05% to about 0.4%, most particularly from about 0.008% to about 0.3%, by weight of the total polymerisation reaction mixture. Typical cross-linkers include tripropylene glycol diacrylate, ethylene glycol dimethacrylate, triacrylate, polyethylene glycol-diacrylate (polyethylene glycol (PEG) molecular weight between about 100 and about 4000, for example PEG400 or PEG600), and methylene bis acrylamide. The hydrogel compositions with which this invention is concerned generally comprise, in addition to a cross-linked polymeric network, an aqueous plasticising medium and, optionally, at least one electrolyte, whilst the materials and processing methods used are normally chosen to provide a suitable balance of adhesive and electrical properties for the desired application. One preferred feature of the process of the invention is that, to achieve the desired properties, the final amount of water required in the hydrogel is present in the formulation prior to gellation, i.e. preferably no water is removed from the hydrogel after manufacture and less than about 10% during manufacture.

As described in more detail below (see the section headed “Polymerisation Conditions”), he process of the invention involves free radical polymerisation and the use of a photoinitiator or a combination of photo- and other initiation. Preferably the reaction mixture comprises an amount of photoinitiator of from about 0.003% to about 0.05%, and particularly from about 0.003% to about 0.04%, most preferably from about 0.009% to about 0.02%, by weight of the total polymerisation reaction mixture. Preferred photoinitiators include any of the following either alone or in combination:

Type I-α-hydroxy-ketones and benzilidimethyl-ketals e.g. Irgacure 65. These are believed on irradiation to form benzoyl radicals that initiate polymerisation. Photoinitiators of this type that are preferred are those that do not catty substituents in the para position of the aromatic ring. Examples include Irgacure 184 and Daracur 1173 as marketed by Ciba Chemicals, as well as combinations thereof.

Photoinitiators of the following general formula are preferred:

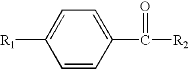

where R₁ can be any of the following: hydrogen, H₃C—S—,

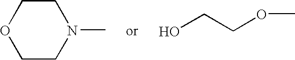

R₁ is most preferably hydrogen

R₂ can suitably be any of the following:

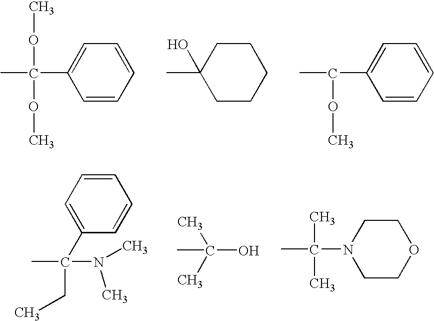

R₂ is most preferably as follows:—

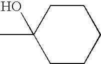

A particularly preferred photoinitiator is 1-hydroxycyclohexyl phenyl ketone, for example, as marketed under the trade name Irgacure 184 by Ciba Speciality Chemicals. Also preferred are Daracur 1173 (2-hydroxy-2-propyl phenyl ketone) and mites of Irgacure 184 and Daracur 1173.

Applications

The compositions described herein may suitably be used in a range of skin contact or covering applications where the composition is brought into contact either with skin or with an intermediary article which interfaces between the composition and the skin. The composition may be unsupported or supported on a backing structure. The compositions may suitably be in the form of sheets, coatings, membranes, composites or laminates. Such applications include patches, tapes, bandages, devices and dressings of general utility or for specific uses, including without imitation biomedical, skin care, personal and body care, palliative and veterinary uses such as, for example, skin electrodes; wound and burn healing; wound and burn management; skin cooling; skin moisturising; skin warming; aroma release or delivery; decongestant release or delivery; pharmaceutical and drug release or delivery; perfume release or delivery; fragrance release or delivery, scent release or delivery; adhesive use, e.g. in skin contacting devices, ostomy and related incontinence devices, and the like. In some fields of application, such as, for example, pharmaceutical delivery devices for the delivery of pharmaceuticals or other active agents to or through mammalian skin, the compositions may optionally contain topical, transdermal or iontophoretic agents and excipients. The compositions may contain penetration-enhancing agents to assist the delivery of water of active agents into the skin. Non-limiting examples of penetration-enhancing agents for use in such applications include methyl oleic acid, isopropyl myristate, Azone® Transcutol® and N-methyl pyrrolidone.

The compositions prepared according to the present invention are used in these applications in generally conventional manner, as will be readily understood by those skilled in this art.

Biomedical Skin Electrodes

A particularly preferred application is in the field of biomedical skin electrodes.

When the hydrogels are intended for use in conjunction with Ag/AgCl medical electrodes, chloride ions are required to be present in order for the electrode to function. Potassium chloride and sodium chloride are commonly used. However any compound capable of donating chloride ions to the system may be used, for example, lithium chloride, calcium chloride, ammonium chloride. The amount that should be added is, dependent on the electrical properties required and is typically about 0.5-8% by weight

In general an electrolyte (e.g. a salt) will need to be included in the polymerisation reaction mixture in appropriate amounts, when the process is used to manufacture a hydrogel composition for use in an electrode.

The compositions prepared according to the present invention are used in biomedical electrodes in generally conventional manner, as will be readily understood by those skilled in this art.

Plasticiser

In one embodiment of the invention the one or more organic plasticiser, when present, may suitably comprise any of the following either alone or in combination: at least one polyhydric alcohol (such as glycerol), at least one ester derived therefrom, at least one polymeric alcohol (such as polyethylene oxide) and/or at least one mono- or poly-alkylated derivative of a polymeric alcohol (such as alkylated polyethylene glycol). Glycerol is the preferred plasticiser. An alternative preferred plasticiser is the ester derived from boric acid and glycerol. When present, the organic plasticiser may comprise up to about 45% by weight of the polymerisation reaction mixture.

Surfactant

Any compatible surfactant may optionally be used as an additional ingredient of the polymerisation mixture. Surfactants can lower the surface tension of the mixture before polymerisation and thus aid processing. Non-ionic, anionic and cationic surfactants are preferred. The surfactant ideally comprises any of the surfactants listed below either alone or in combination with each other and/or with other surfactants. The total amount of surfactant, if present, is suitably up to about 10% by weight of the total polymerization reaction mixture, preferably from about 0.05% to about 2% by weight, more preferably from about 0.05% to about 1% by weight.

1. Non-Ionic Surfactants

Suitable non-ionic surfactants include, but are not limited to, those selected from the group consisting of the condensation products of a higher aliphatic alcohol such as a fatty alcohol containing about 8 to about 20 carbon atoms, in a straight or branched chain configuration, condensed with about 3 to about 100 moles, preferably about 5 to about 40 moles and most preferably about 5 to about 20 moles of ethylene oxide.

Examples of such non-ionic ethoxylated fatty alcohol surfactants are the Tergitol™ 15-S series from Union. Carbide and Brij™ surfactants from ICI. Tergitol™ 15-S surfactants include C₁₁-C₁₅ secondary alcohol polyethyleneglycol ethers. Brij™ 58 surfactant is polyoxyethylene(20) cetyl ether, and Brij™ 76 surfactant is polyoxyethylene (10) stearyl ether.

Other suitable non-ionic surfactants include, but are not limited to, those selected from the group consisting of the polyethylene oxide condensates of one mole of alkyl phenol containing from about 6 to 12 carbon atoms in a straight or branched chain configuration, with about 3 to about 100 moles of ethylene oxide. Examples of non-ionic surfactants are the Igepal™ CO and CA series from Rhone-Poulenc. Igepal™ CO surfactants include nonylphenoxy poly(ethyleneoxy) ethanols. Igepal™ CA surfactants include octylphenoxy poly(ethyloneoxy)ethanols.

Another group of usable non-ionic surfactants include, but are not limited to, those selected from the group consisting of block copolymers of ethylene oxide and propylene oxide or butylene oxide. Examples of such non-ionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers. The balance of hydrophobic and hydrophilic components within the surfactant together with the molecular weight are found to be important. Suitable examples are Pluronic L68 and Tetronic 1907. Particularly suitable examples are Pluronic L64 and Tetronic 1107.

Still other satisfactory non-ionic surfactants include, but are not limited to, those selected from the group consisting of sorbitan fatty acid esters, polyoxyethylene sorbitan fatty acid esters and polyoxyethylene stearates. Examples of such fatty acid ester non-ionic surfactants are the Span™, Tween™, and Myrj™ surfactants from ICI, Span™ surfactants include C₁₂-C₁₈ sorbitan monoesters. Tween™ surfactants include poly(ethylene oxide) C₁₂-C₁₈ sorbitan monoesters. Myrj™ surfactants include poly(ethylene oxide) stearates.

2. Anionic Surfactants

Anionic surfactants normally include a hydrophobic moiety selected from the group consisting of (about C₆ to about C₂₀) alkyl alkylaryl and alkenyl groups and an anionic group selected from the group consisting of sulfate, sulfonate, phosphate, polyoxyethylene sulfate, polyoxyethylene sulfonate, polyoxyethylene phosphate and the alkali metal salts, ammonium salts, and tertiary amino salts of such anionic groups.

Anionic surfactants which can be used in the present invention include, but are not limited to those selected from the group consisting of (about C₆ to about C₂₀) all or alkylaryl sulfates or sulfonates such as sodium lauryl sulfate (commercially available as Polystep™ B-3 from Srepan Co.) and sodium dodecyl benzene sulfonate, (commercially available as Siponsate™ DS-10 from Rhone-Poulenc); polyoxyethylene (about C₆ to about C₂₀) alkyl or alkylphenol ether sulfates with the ethylene oxide repeating unit in the surfactant below about 30 units, preferably below about 20 units, most preferably below about 15 units, such as Polystep™ B-1 commercially available from Stepan Co. and Alipal™ EP110 and 115 from Rhone-Poulenc; (about C₆ to about C₂₀) alkyl or alkylphenoxy poly (ethyleneoxy)ethyl mono-esters and di-esters of phosphoric acid and its salts, with the ethylene oxide repeating unit in the surfactant below about 30 units, preferably below about 20 units, most preferably below about 15 units, such as Gafac™RE-510 and Gafac™ RE-610 from GAF.

3. Cationic Surfactants

Cationic surfactants useful in the present invention include, but are not limited to, those selected from the group consisting of quaternary ammonium salts in which at least one higher molecular weight group and two or tee lower molecular weight groups are linked to a common nitrogen atom to produce a cation, and wherein the electrically-balancing anion is selected from the group consisting of a halide (bromide, chloride, etc.), acetate, nitrite, and lower alkosulfate (methosulfate etc,). The higher molecular weight substituent(s) on the nitrogen is/are often (a) higher alkyl group(s), containing about 10 to about 20 carbon atoms, and the lower molecular weight substituents maybe lower alkyl of about 1 to about 4 carbon atoms, such as methyl or ethyl, which may be substituted, as with hydroxy, in some instances. One or more of the substituents may include a aryl moiety or may be replaced by an aryl, such as benzyl or phenyl.

In a preferred embodiment of the invention the surfactant comprises at least one propylene oxide/ethylene oxide block copolymer, for example such as that supplied by BASF Plc under the trade name Pluronic P65 or L64.

Polymerisation Conditions

In preparing hydrogel compositions in accordance with the invention, the ingredients will be mixed to provide a reaction mixture in the form of an initial pre-gel aqueous based liquid formulation, and this is then converted into a hydrogel by a free radical polymerisation reaction. Photo-polymerisation may be achieved using photoinitiators, optionally together with other initiators, such as heat and/or ionizing radiation. Photoinitiation will usually be applied by subjecting the pre gel reaction mixture containing an appropriate photoinitiation agent to ultraviolet (UV) light after it has been spread or coated as a layer on siliconised release paper or other solid substrate. The incident UV intensity, at a wavelers in the range from 240 to 420 nm, is typically greater than about: 10 mW/cm². The processing will generally be carried out in a controlled manner involving a precise predetermined sequence of mixing and thermal treatment or history.

The UV irradiation time scale should ideally be less than 60 seconds, and preferably less than 10 seconds to form a gel with better than 95% conversion of the monomers. Those skilled in the art will appreciate that the extent of irradiation will be dependent on a number of factors, including the UV intensity, the type of UV source used, the photoinitiator quantum yield, the amount of monomer present, the nature of the monomer(s) present, the presence of dissolved oxygen, the presence of polymerisation inhibitor, the thickness of the reaction mixture when coated onto the substrate and the nature of substrate onto which the reaction mixture is coated.

Other Additives

Additional polymer(s), typically rheology modifying polymer(s), may be incorporated into the polymerisation reaction mixture at levels typically up to about 10% by weight of total polymerisation reaction mixture, e.g. from about 0.2% to about 10% by weight. Such polymer(s) may include polyacrylamide, poly-NaAMPS, polyethylene glycol (PBG), polyvinylpyrrolidone (PVP) or carboxymethyl cellulose.

Additional functional ingredients may also incorporated in the reaction mixture used in the invention, including antimicrobial agents (e.g. citric acid, stannous chloride) and, for drug delivery applications, pharmaceutically active agents, the latter being designed to be delivered either passively (e.g. transdermally) or actively (e.g. iontophoretically) through the skin. For this purpose, penetration enhancing agents may also be presents in the reaction mixture and resultant hydrogel, as described above in the section headed “Applications”.

### EXAMPLES OF THE INVENTION

The invention will be further descried with reference to the following Examples, which should not be understood to limit the scope of the invention

### Example 1

400 g of a 50% aqueous solution of sodium-2-acrylamido-2-methylpropane sulphonate (NaAMPS, Lubrizol) was added to 450 g water. 150 g glycerol W added and the mixture stirred for 30 minutes. A solution of crosslinker and photoinitiator was made by adding 2.3 g of IRR280 (PEG400 diacrylate, UCB Chemicals) to 0.012 g of photoinitiator, Daracur 1173 (Ciba Specialty Chemicals). This was added to the mixture, which was stirred for 1 hour, covered to exclude light. 50 g of the sure at a coat weight of 1.5 kg/sq.m was in the laboratory on a tray lined with siliconised paper by passing at a speed of 7 m/minute three times under ultra-violet (UV) radiation of −80 W/cm from a medium pressure mercury vapour lamp. The cured gel was covered with a siliconised high density polyethylene (HDPE) top liner.

### Examples 2 to 8

Gels having various photoinitiator levels were made by the procedure described in Example 1 above, substituting the 0.012 g of photoinitator by the amounts shown in Table 1.

TABLE 1

| Example No. | 1 | 2 | 3 | 4 | 5 | 6 | 7 | 8 |
|---|---|---|---|---|---|---|---|---|
| Daracur | 0.012 | 0.035 | 0.115 | 0.23 | 0.345 | 0.46 | 0.575 | 0.69 |
| 1173 (g) | | | | | | | | |

Examples 2, 3, 4, 5 and 6 gave acceptable materials; 1, 7 and 8 did not

### Examples 9 to 16

Formulations were prepared containing the following parts by weight as shown in Table 2.

TABLE 2

| Example No. | 9 | 10 | 11 | 12 | 13 | 14 | 15 | 16 |
|---|---|---|---|---|---|---|---|---|
| 50% NaAMPS | 40 | 57 | 45 | 36 | 50 | 40 | 57 | 57 |
| solution | | | | | | | | |
| Water | 60 | 43 | 38 | 64 | 50 | 60 | 43 | 43 |
| Pluronic P65 | 0.18 | 0.18 | 0.18 | 0.18 | 0.18 | 0.18 | 0.18 | 0.18 |
| (surfactant) | | | | | | | | |
| Polyacrylamide | — | — | 17 | — | — | — | — | — |
| (12% solution) | | | | | | | | |
| Chlorohexidine | — | — | — | — | — | — | — | 0.5 |
| Gluconate | | | | | | | | |
| Potassium | — | — | — | — | — | 5 | 4 | — |
| Chloride | | | | | | | | |
| IRR280 | 0.29 | 0.19 | 0.238 | 0.29 | 0.143 | 0.29 | 0.29 | 0.29 |
| Daracur.1173 | 0.01 | 0.01 | 0.01 | 0.01 | 0.007 | 0.01 | 0.01 | 0.01 |

Polyacrylamide solution available as Glascol W17™ Ciba Specialty Chemicals Chlorohexidine Gluconate and Potassium Chloride from Aldrich Pluronic P65 from BASF

Examples 9-16 gave acceptable hydrogels.

1. A process for the manufacture of a high water content cross-linked hydrogel composition, comprising:
(i) preparing a m re comprising:
(1) one or more unsaturated free radically photo-polymerisable monomer capable of polymerisation to a hydrophilic polymer,
(2) one or more free radical photoinitiator;
(3) one or more cross-linking agent comprising a multifunctional unsaturated free radically photo-polymerisable compound; and
(4) water; and
(ii) irradiating the mixture with light of sufficient intensity and at an appropriate wavelength to polymerise and cross-link the mixture to form the composition;
wherein substantially all of components (1) to (4) present in the mixture in step (i) are also present in the composition resulting from step (ii), the photoinitiator (2) is present, in the mixture in step (i) in an amount between about 0.002% and about 0.05% by weight of the total mixture, and the cross-linking agent (3) is present in the mixture in step (i) in an amount less than about 0.5% by weight of the total

2. A process according to claim 1, wherein the resultant hydrogel composition comprises-more than about 50% by weight of water.

3. A process according to claim 2, wherein the resultant hydrogel composition comprises between about 60% and about 95% by weight of water.

4. A process according to any one of the preceding claim, wherein the mixture prepared in step (i) further comprises one or more electrolyte.

5. A process according to any one of the preceding claims, wherein the mixture prepared in step (i) further comprises one or more organic plasticiser.

6. A process according to claim 5, wherein the organic plasticiser comprises any of the following either alone or in combination: at least one polyhydric alcohol at least one ester derived therefrom, at least one polymeric alcohol and/or at least one mono- or poly-alkylated derivative of a polymeric alcohol.

7. A process according to claim 5 or 6, wherein organic plasticiser comprises glycerol or an ester derived from boric acid and glycerol.

8. A process according to any one of the preceding claims, wherein the mixture prepared in step (i) further comprises one or more surfactant.

9. A process according to claim 1, wherein the mixture prepared in step (i) consists essentially of components (1) to (4) and optionally one or more electrolyte and/or one or more organic plasticiser and/or one or more surfactant.

10. A process according to any one of the preceding claims, when carried out in a continuous manner for the production of a continuous hydrogel sheet.

11. A process according to claim 10, wherein the hydrogel sheet has a thickness in the range of about 0.2 mm to about 2 mm.

12. A process according to any one of the preceding claims, wherein the one or more monomer comprises 2-acrylamido-2-methylpropane sulphonic acid, acrylic acid, acrylic acid (3-sulphopropyl) ester, a substituted derivative thereof, or a salt thereof.

13. A process according to any one of the preceding claims, wherein the one or more monomer comprises at least one of acrylamide or a mono- or di-N-alkylacrylamide or an analogue thereof containing an alkyl or substituted alkyl group linked to a carbon-carbon double bond via an amido or alkylamido function.

14. A process according to claim 13, wherein the analogue is diacetone acrylamide, a vinyl lactam, an N-alkylated acrylamide, an N,N-dialkylated acrylamide, N-vinyl pyrrolidone or acryloyl morpholine.

15. A process according to any one of the, preceding claims, wherein the photoinitiator (2) is present in the mixture in step (i) in an amount of from about 0.003% to about 0.05% by weight of the total polymerisation reaction mixture.

16. A process according to claim 15, wherein the photoinitiator (2) is present in the mixture in step (i) in an amount of from about 0.003% to about 0.04% by weight of the total polymerisation reaction mixture.

17. A process according to claim 15, wherein the photoinitiator (2) is present in the mixture in step (i) in an amount of from about 0.009% to about 0.02% by weight of the total polymerisation reaction mixture.

18. A process according to any one of the preceding claim, wherein the photoinitiator comprises a compound of the following general formula: 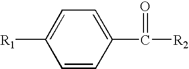
where R₁, is selected from hydrogen H₃C—S—, 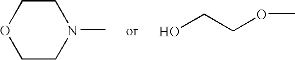
R₂ is selected from

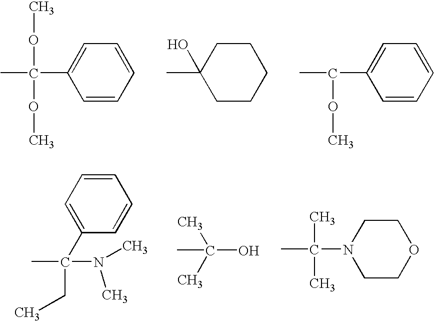

19. A process according to claim 18, wherein R₁ is hydrogen.

20. A process according to claim 18 or 19, wherein R₂ is

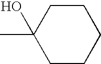

21. A process according to any one of the preceding claims, wherein the one or more cross-linking agent (3) is present in the mixture in step (i) in an amount of from about 0.01% to about 0.5% by weight of the total polymerisation reaction mixture.

22. A process according to claim 21, wherein the cross-linking agent (3) is present in the mixture in step (i) in an amount of from about 0.05% to about 0.4% by weight of the total polymerisation reaction mixture.

23. A process according to claim 21, wherein the cross-ling agent (3) is present in the mixture in step (i) in an amount of from about 0.08% to about 0.3% by weight of the total polymerisation reaction mixture.

24. A process according to any one of the preceding claims, wherein the cross-linking agent comprises tripropylene glycol diacrylate, ethylene glycol dimethacrylate, triacrylate, polyethylene glycol diacrylate or methylene bis acrylamide.

25. Use of a controlled amount of one or more free radical photoinitiator in the range between about 0.002% and about 0.05% by weight of the total mixture and a controlled amount of one or more multifunctional unsaturated free radically photo-polymerisable cross-linking agent in the range less than about 0.5% by weight of the total mixture, in a pre-photopolymerisation mixture comprising also water and one or more unsaturated free radically photo-polymerisable monomer in a process for the manufacture of a high water content cross-linked hydrogel composition having a desired predetermined combination of two or more of: non-greasy feel to human skin, structural integrity, low brittleness and good industrial processability in sheet form.

26. A use according to claim 25, wherein the process is as claimed in any one of claims 1 to 24.

27. A hydrogel composition prepared by a process according to any one of claims 1 to 24 or by a use according to claims 25 or 26.

28. A product for contacting or covering the skin, comprising a hydrogel composition according to claim 27.

29. A product according to claim 28, dimensioned and configured as an article selected from patches, tapes, bandages, devices and dressings of general utility or for specific biomedical, skin care, personal care, body care, palliative or veterinary uses.

30. A product according to claim 29, having a utility selected from: skin electrodes; wound and bum healing, wound and burn management; skin cooling; skin moisturising; skin warming; aroma release or delivery; decongestant release or delivery; pharmaceutical and drug release or delivery; deliver, and adhesive use in skin contacting devices, ostomy and related incontinence devices.
